(19) United States
(12) Patent Application Publication
(10) Pub. No.: US 2026/0043784 A1
(43) Pub. Date: Feb. 12, 2026

(54) PETROPHYSICAL MODEL GENERATION AND USES THEREOF

(71) Applicant: SAUDI ARABIAN OIL COMPANY, Dhahran (SA)

(72) Inventors: Jun GAO, Dhahran (SA); Xupeng HE, Dhahran (SA); Hyung KWAK, Dhahran (SA)

(73) Assignee: SAUDI ARABIAN OIL COMPANY, Dhahran (SA)

(21) Appl. No.: 18/799,475

(22) Filed: Aug. 9, 2024

(51) Int. Cl. G01N 33/24; G01N 23/046; G01N 24/08; G16C 60/00

(52) U.S. Cl. CPC G01N 33/24; G01N 23/046; G01N 24/081; G16C 60/00; G01N 2223/419; G01N 2223/606; G01N 2223/635; G01N 2223/649

(57) ABSTRACT

Systems and methods are disclosed relating to reservoir characterization. A computed tomography (CT) imaging device is used to generate a CT image of a rock sample from a reservoir and segmented into CT slices. The CT slices are processed to identify textures of the rock sample to provide texture data. The rock sample is scanned using nuclear magnetic resonance (NMR) to provide NMR data. The NMR data is segmented to provide NMR segments. The NMR segments and texture data are analyzed to determine a contribution of each texture in each CT slice to one or more relaxation times in a corresponding NMR segment for each CT slice. A petrophysical property is predicted for each texture of each CT slice based on a contribution of each texture and the corresponding NMR segment for each CT slice. A petrophysical model for the reservoir is generated based on the predicted petrophysical property.

### FIELD OF THE DISCLOSURE

This disclosure relates generally to reservoir characterization.

### BACKGROUND OF THE DISCLOSURE

Carbonate reservoir rocks are specific types of sedimentary rock formations composed primarily of carbonate minerals such as calcite, aragonite, and dolomite. Carbonate reservoirs are significant sources of hydrocarbons, containing oil and gas trapped within pore spaces and fractures of a rock matrix. A porosity and permeability of carbonate rocks makes such rocks suitable for storing and transmitting hydrocarbon fluids. Carbonate reservoir rocks are known to exhibit heterogeneity. Heterogeneity refers to a variability in physical properties, composition, and/or pore structure that impacts fluid flow behavior and permeability at microscopic and macroscopic scales. For example, at a microscopic scale, small-scale features within the rock can be observed at a level of individual mineral grains, pores, and other microscopic constituents. At this scale, variations in pore size, shape, connectivity, and/or distribution are factors (referred to as microscopic features) that influence fluid behavior of fluids in rock. For example in a reservoir formed from carbonate rock, the fluid behavior can characterize how oil, water, or gas moves through microscopic pore spaces, including how easily fluids mix or separate, how such fluids respond to pressure changes, and how the fluids interact with a surface of the carbonate rock. Permeability determines how easily oil or gas can be extracted from the reservoir. At a macroscopic scale, larger-scale features within carbonate rock, such as vugs (cavities), fractures, and/or other structural elements (referred to as macroscopic features) can be observed with a naked eye or through imaging techniques like computer tomography (CT) and also can impact fluid flow and permeability of the carbonate rock.

Proper representation (or characterization) of a pore structure of carbonate rock (e.g., is needed in order to accurately determine an impact that a pore structure of the carbonate rock has on fluid behavior so that fluid flow patterns can be accurately predicted. Permeability can be estimated using models that incorporate pore size distribution, shape, and connectivity data based on techniques like mercury injection capillary pressure (MICP) analysis and imaging methods (e.g., CT scanning). MICP analysis is a laboratory method that is used to examine void spaces (pores) within a rock sample (e.g., carbonate rock sample). The MICP method is used to identify the microscopic features so that a pore size distribution of the rock sample can be determined. For example, the rock sample can be saturated with mercury under increasing pressure, causing the mercury to invade pore spaces of the rock sample. As the pressure is gradually increased, mercury fills progressively smaller pores, allowing users to measure a volume of mercury intruded at each pressure increment. Thus, the mercury starts filling larger pores at lower pressures and then progressively fills smaller and smaller pores as the pressure increases. Carbonate reservoir rocks typically exhibit a broader range of pore sizes (and thus has a wider pore size distribution) when compared to sandstones, and encompass both interparticle microporosity and intercrystallite microporosity. Interparticle microporosity refers to pores located between particles that make up a rock. Intercrystallite microporosity refers to pores located between individual crystals within the rock. Thus, the pore-size distribution provides information about pore sizes within a rock, such a range and frequency of different pore sizes of the rock (e.g., how pores are spread throughout the rock), and how much of a volume of the rock is occupied by the pores.

In some examples, to detect larger structures present in a core sample reservoir imaging techniques such as CT can be used to provide detailed visualizations of the macroscopic features (e.g., vugs (cavities), fractures, and various textures. These larger-scale features also contribute to macroscopic heterogeneity of carbonate rock. For example, vugs and fractures create pathways for fluids to move through the carbonate rock. Fractures, for example, enhance permeability and fluid flow within a reservoir by providing conduits for hydrocarbons to migrate from a source rock to production wells. Different textures within a core plug (e.g., a smaller, cylindrical section of the whole core that is extracted for laboratory testing) and whole cores (e.g., a complete intact cylindrical section of carbonate rock retrieved from a subsurface), such as grain size variations or sedimentary structures, also affects fluid flow behavior. For example, some textures can enhance permeability, while others can act as barriers to fluid flow, leading to preferential flow paths or flow barriers within the carbonate reservoir rock. A texture in a rock refers to physical characteristics of the rock, for example, grain size, shape, orientation, and/or arrangement of grains and pores within a rock matrix of the rock.

Once a columnar rock sample of a rock has been taken from the well (the core), the core can undergo core analysis. In some instances, core samples are collected from a wellbore (the well) using a specialized coring tool during drilling operations. These samples can be retrieved from different depths to capture variations in lithology, porosity and fluid content. Cores are extracted from a subsurface and brought to a surface in sections while maintaining an orientation and integrity.

Upon retrieval of the core, core plugs (core samples) can be extracted from the core for core analysis. In some examples, the extracted core plugs are screened to avoid selecting heterogeneous samples for further analysis. Heterogeneous samples may not accurately represent overall properties of the reservoir. For example, laboratories select homogeneous or less heterogeneous samples, so that data can be obtained that more accurately reflects average characteristics of the reservoir rock. Traditionally, visual inspection has been relied upon to identify variations in lithology, texture, fractures, and other features as indicators of heterogeneity within the core. Visual inspection allows a laboratory to identify macroscopic features (e.g., such as lithology variations, fractures, and textures). By visually screening core plugs, laboratories can select representative samples for further analysis such as petrophysical measurements, ensuring that data obtained accurately reflects reservoir's properties. Thus, existing laboratory techniques fail to account for heterogeneity within these samples due to an assumption that screened core plugs are to exhibit uniform porosity and permeability characteristics.

### SUMMARY OF THE DISCLOSURE

Various details of the present disclosure are hereinafter summarized to provide a basic understanding. This summary is not an extensive overview of the disclosure and is neither intended to identify certain elements of the disclosure nor to delineate the scope thereof. Rather, the primary purpose of this summary is to present some concepts of the disclosure in a simplified form prior to the more detailed description that is presented hereinafter.

According to an embodiment, a method can include imaging, using a computed tomography (CT) imaging device to generate a CT image of a rock sample from a reservoir, segmenting the CT image into CT slices, processing the CT slices using a texture classifier to identify each texture present in each CT slice of the CT slices to provide texture data for each CT slice, scanning the rock sample using an nuclear magnetic resonance (NMR) device to provide NMR data for the rock sample, the NMR data characterizing relaxation times across different regions of the rock sample reflecting variations in pore sizes within the rock sample, segmenting the NMR data into intervals corresponding to a scanning interval of a portion of the rock sample by the NMR device to provide NMR segments, analyzing the NMR segments and the texture data to determine a contribution of each texture in each CT slice to one or more relaxation times in a corresponding NMR segment of the NMR segments for each CT slice, predicting one or more petrophysical properties for each texture of each CT slice based on a determined contribution of each texture and the corresponding NMR segment for each CT slice, and generating a petrophysical model for the reservoir based on the predicted one or more petrophysical properties for each texture of each CT slice.

According to another embodiment, a system can include one or more computing platforms configured to: segment a CT image of a rock sample from a reservoir into CT slices, process the CT slices using a texture classifier to identify each texture present in each CT slice of the CT slices to provide texture data for each CT slice, receive NMR data generated from an NMR scan of the rock sample, the NMR data characterizing relaxation times across different regions of the rock sample reflecting variations in pore sizes within the rock sample, segment the NMR data into intervals corresponding to a scanning interval of a portion of the rock sample scanned by an NMR device to provide NMR segments, analyze the NMR segments and the texture data to determine a contribution of each texture in each CT slice to one or more relaxation times in a corresponding NMR segment of the NMR segments for each CT slice, determine one or more petrophysical properties for each texture of each CT slice based on a determined contribution of each texture and the corresponding NMR segment for each CT slice, and generate a petrophysical model for the reservoir based on the predicted one or more petrophysical properties for each texture of each CT slice.

In yet another embodiment, a system can include memory to store machine-readable instructions and one or more processors to access the memory and execute the machine-readable instructions, the machine-readable instructions can include a CT image segmentor to segment a CT image of a rock sample from a reservoir into CT slices, a texture classifier to identify each texture present in each CT slice of the CT slices to provide texture data for each CT slice, an NMR image segmentor to segment NMR data into intervals corresponding to a scanning interval of a portion of the rock sample scanned by the NMR device to provide NMR segments, a factor analyzer to analyze the NMR segments and the texture data to determine a contribution of each texture in each CT slice to one or more relaxation times in a corresponding NMR segment of the NMR segments for each CT slice, a calculator to determine one or more petrophysical properties for each texture of each CT slice based on a determined contribution of each texture and the corresponding NMR segment for each CT slice, and a model generator to generate a petrophysical model for the reservoir based on the predicted one or more petrophysical properties for each texture of each CT slice.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 is an example of a block diagram of a system for petrophysical model generation.

FIG. 2 is an example of a method for generating a petrophysical model for a reservoir.

FIG. 3 is an example of another method for generating a petrophysical model for a reservoir.

FIG. 4 is a block diagram of a system that can be used to perform one or more methods according to an aspect of the present disclosure.

FIG. 5 is an example of a cloud computing environment that can be used to perform one or more methods according to an aspect of the present disclosure.

### DETAILED DESCRIPTION

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Examples are disclosed herein relating to characterization of a rock for hydrocarbon application and/or use thereof. To estimate permeability and porosity, three-dimensional (3D) images from micro-computed tomography (micro-CT) scans of core plugs are used. Core plugs, also known as core samples, are extracted from a reservoir and processed in a laboratory to estimate petrophysical properties such as porosity and permeability, especially for reservoir rocks like carbonates. Micro-CT technology is employed to capture high-resolution 3D images of microscopic features, such as the pore structure within the rock matrix, a practice known as digital rock physics. These 3D images, which capture the pores of a core plug at a submicron level, are utilized to estimate the porosity and permeability of the core plug.

However, achieving submicron resolution across an entire sample volume for a typical 1.5″ diameter core plug using micro-CT is not feasible. Submicron resolution requires extremely high precision and substantial computational resources, making it both time-consuming and costly. Additionally, simulating fluid behavior within the pore network of a core plug necessitates detailed knowledge of its pore-scale structure. Conducting such simulations at fine scales demands extensive computational resources and time, making the process complex. High-resolution imaging techniques like micro-CT capture pore-scale features in fine detail but fail to provide a comprehensive view of larger-scale heterogeneity present in the reservoir. Achieving high resolution typically requires focusing on a smaller area or volume of the core sample, which limits the field of view (FOV). The FOV refers to the extent of the core sample that can be imaged in a single scan. High resolution is essential for accurately capturing intricate pore structures present in carbonate reservoir rocks.

High-resolution imaging techniques like micro-CT capture pore-scale features in fine detail but fail to provide a comprehensive view of larger-scale heterogeneity present in the reservoir. Achieving high resolution typically requires focusing on a smaller area or volume of the core sample, which limits the field of view (FOV). The FOV refers to the extent of the core sample that can be imaged in a single scan. High resolution is essential for accurately capturing intricate pore structures present in carbonate reservoir rocks.

There is a trade-off between resolution and FOV: increasing resolution to capture fine-scale details results in a narrower FOV, limiting the ability to capture the full extent of heterogeneity across different scales. Conversely, widening the FOV to encompass larger areas sacrifices resolution, making it difficult to accurately resolve pore-scale features. This trade-off poses a significant technical challenge for laboratories in accurately characterizing carbonate reservoirs, as it is difficult to reconcile detailed information obtained at a pore scale with broader reservoir-scale properties.

As a result, laboratories often resort to larger-scale heterogeneity observations from whole core scans using micro-CT. Whole core scans offer a broader FOV, allowing for imaging of larger portions of the core sample at lower resolutions. While this approach sacrifices some detail at the pore scale, it provides a more comprehensive view of rock heterogeneity.

Once the micro-CT images have been obtained, these 3D images are processed using computational techniques, which include segmentation and classification. For example, the 3D image of the core plug can be segmented into segmented core samples. Segmenting core samples into segments allows laboratories to assume that rocks with similar textures possess comparable petrophysical properties. The 3D image of the core plug can be received or retrieved, containing information about texture variations within the core plug. The 3D image can be processed by a convolutional neural network (CNN) for texture classification, which involves analyzing features of the 3D image to categorize different regions of the core plug based on corresponding textures (e.g., physical attributes and/or features of a rock, such as shape, size, arrangement, and distribution of grains within the rock). In some examples, the CNN is based on a U-Net architecture used for image segmentation tasks.

After identifying the regions, further scanning is performed at a finer scale within these regions of interest. This finer-scale scanning provides more detailed data (finer-scale data) about the pore structure and properties associated with each texture type. Detailed pore structure data is used to create a pore network model for each texture type. Each model represents the spatial arrangement and connectivity of the pores within each identified region of the core plug. The pore network model can be used to simulate fluid flow and calculate permeability at the microscale level using techniques like the Lattice Boltzmann Method (LBM).

Using the pore network model, the permeability of the core plug can be estimated (or determined) using the Lattice Boltzmann Method (LBM). The LBM is a computational technique used to simulate fluid flow within porous media at a microscale level. By applying this method to the representative textures identified in the core sample (of the pore network model), permeability values associated with each texture type can be calculated. The porosity of the core plug is determined by analyzing the volume fraction of the pore spaces within each identified texture type, using the detailed pore structure data obtained from the micro-CT scans.

The permeability and porosity values obtained from the LBM and pore structure analysis can then be used to construct a continuum model, such as a Darcy-scale model. A continuum model is a mathematical or computational representation of a physical system that treats the system as a continuous medium. This model assumes that properties such as density, porosity, permeability, temperature, and/or pressure vary smoothly and continuously throughout a domain, rather than being discrete or isolated at specific points. In the context of porous media, such as rock formations, a continuum model averages these properties over a larger scale, providing a simplified yet practical approach to describing the behavior of the system. Thus, the Darcy-scale model provides an approximation of an overall permeability and porosity behavior of the core sample at a larger scale, suitable for reservoir engineering applications and simulations. The continuum model uses permeability and porosity values derived from pore network models to create a larger-scale, simplified representation of the core sample (of the reservoir). The continuum model can be used for practical simulations and engineering applications.

Examples are disclosed herein for determining permeability and porosity based on nuclear magnetic resonance (NMR) measurements. In some examples, a deep learning algorithm (e.g., a CNN algorithm) for rock texture classification is implemented using 3D images, and can be used in combination with NMR porosity and permeability. By integrating data from both sources, continuum porosity and/or permeability models can be derived. These models can serve to enhance an interpretation of reservoir properties, particularly in a presence of core-scale heterogeneity. Additionally, the combined use of advanced analytical techniques, such as deep learning algorithms, alongside NMR measurements, allows for a more comprehensive understanding of the reservoir's characteristics. This integrated approach augments other measurements, including core analysis data and well logs, resulting in a more precise interpretation of reservoir behavior.

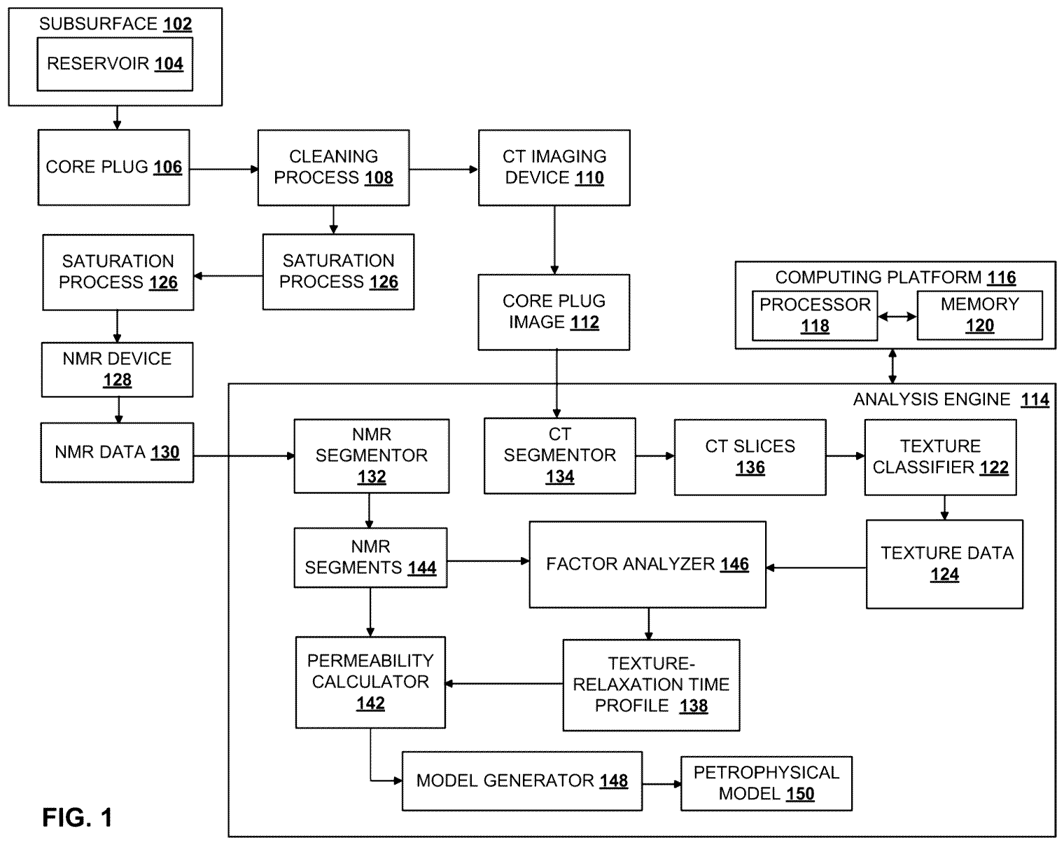

FIG. 1 is an example of a block diagram of a system 100 for petrophysical model generation, such as a petrophysical model 150 that can represent one or more petrophysical properties of a reservoir 104 of a subsurface 102. The one or more petrophysical properties can include a permeability and/or porosity. The reservoir 104 can be a rock formation, which can contain hydrocarbons, such as oil or natural gas, which can be trapped within its pores. The reservoir 104 can be formed from one or more geological layers, which can include reservoir rock that can act as a storage space for the hydrocarbons. The reservoir rock can be a carbonate rock, in some instances referred to as a reservoir carbonate rock. In some instances, a well is drilled into the reservoir to provide access to the hydrocarbons trapped within the reservoir 104.

For example, a core plug 106 can be extracted from the reservoir 104, which includes at least portion of the reservoir rock. One or more drilling operations can be performed so that a cylindrical section of the reservoir rock, known as a core, can be extracted from the subsurface 102 using a coring tool. Once the core plug 106 is retrieved, the core plug 106 can undergo a cleaning process 108 to remove any drilling mud, debris, and/or other contaminants that can be present on its surface. In a non-limiting example, the core plug 106 can measure about 1.5 inches (or 38.1 mm) in diameter and about 2 inches (or 50.8 mm) in length.

The core plug 106, after cleaning, can be imaged using a CT imaging device 110. In some examples, the CT imaging device 110 is a microCT scanner. The CT imaging device 110 can be used to generate (create) a core plug image 112 (e.g., a 3D image) of the core plug 106 and thus provide a CT image of the core plug 106 (rock sample). For example, the core plug 106 can be placed inside the microCT scanner. The microCT scanner can rotate around the core plug 106, capturing a series of X-ray images (e.g., 2D radiographs) from multiple angles. The series of X-ray images represent cross-sectional slices of the core plug 106. The microCT scanner can reconstruct a 3D image of the core plug 106 based on the series of X-ray images to provide the core plug image 112. The core plug image 112 can have a DICOM or TIFF format.

The core plug image 112 can include volumetric pixels (voxels) representing a small volume element. Parameters of a scanning process of the core plug 106 implemented by the CT imaging device 110 can be controlled to set a resolution of the scanning process to define a level of detail that is captured of the core plug 106. Scanning parameters, such as voxel size can impact a resolution of the core plug image 112. A resolution refers to a level of detail or sharpness in the core plug image 112, with a higher resolution indicating finer details can be captured. The voxel size can be set to about 10 to about 20 microns per voxel. By setting the resolution to about 10 to about 20 microns per voxel results in sufficient detail, such that microscopic features (e.g., pore structures) of the core plug 106 can be captured in the core plug image 112.

The core plug image 112 can be processed by an analysis engine 114 to determine the one or more petrophysical properties of the reservoir 104. For example, the analysis engine 114 can be implemented using one or more modules, shown in block form in the drawings. The one or more modules can be in software or hardware form, or a combination thereof. In some examples, the analysis engine 114 can be implemented as machine readable instructions for execution on a computing platform 116, as shown in FIG. 1. The computing platform 116 can include one or more computing devices selected from, for example, a desktop computer, a server, a controller, a blade, a mobile phone, a tablet, a laptop, a personal digital assistant (PDA), and the like. The computing platform 116 can include a processor 118 and a memory 120. By way of example, the memory 120 can be implemented, for example, as a non-transitory computer storage medium, such as volatile memory (e.g., random access memory), non-volatile memory (e.g., a hard disk drive, a solid-state drive, a flash memory, or the like), or a combination thereof. The processor 118 can be implemented, for example, as one or more processor cores. The memory 120 can store machine-readable instructions (e.g., the analysis engine 114) that can be retrieved and executed by the processor 118. Each of the processor 118 and the memory 120 can be implemented on a similar or a different computing platform. The computing platform 116 can be implemented in a cloud computing environment (for example, as disclosed herein) and thus on a cloud infrastructure. In such a situation, features of the computing platform 116 can be representative of a single instance of hardware or multiple instances of hardware executing across the multiple of instances (e.g., distributed) of hardware (e.g., computers, routers, memory, processors, or a combination thereof). Alternatively, the computing platform 116 can be implemented on a single dedicated server or workstation. In some examples, the analysis engine 114 can be implemented as part of or integrated into reservoir software or platform, but in other instances, can be implemented as a stand-alone application/software (e.g., and can be invoked by software, a program, a routine, in other instances, invoked by a user).

The analysis engine 114 includes a CT segmentor 134. The CT segmentor 134 can segment the core plug image 112 into CT slices 136 or also known as CT segments. Each CT slice can represent a 2D cross-section of the core plug 106. In some instances, before segmentation, the core plug image 112 (e.g., the microCT image of the core plug 106) can be preprocessed by the analysis engine 114 to enhance contrast and/or remove noise. The CT segmentor 134 can use a segmentation algorithm to partition the core plug image 112 to provide the CT slices 136, for example, based on differences in pixel intensity. Example segmentation algorithms that can be implemented by the CT segmentor 134 can include, but not limited to, K-means clustering, mean shift, watershed transform, graph cut, and region growing.

In some examples, the CT segmentor 134 can segment the core plug image 112 based on segmentation criteria to provide the CT slices 136. The segmentation criteria defines a volume of a portion or segment that is extracted from the core plug image 112 to provide a corresponding CT slice of the CT slices 136. The extracted volume can be measured in cubic millimeters (mm³). In a non-limiting example, the extracted volume can be within a range of about 1 to about 8 mm³. The thickness of the extracted volume can be set to align with (and thus is same or similar to) a thickness of an NMR segment (spatial slice), as disclosed herein.

The analysis engine 114 further includes a texture classifier 122 for classification of the CT slices 136. The texture classifier 122 can correspond to a trained machine learning (ML) algorithm, such as a CNN algorithm. The CNN algorithm can be based on a U-Net architecture. For example, the analysis engine 114 can train the ML algorithm based on a training dataset. The training dataset can include texture characteristics (e.g., groups of texture characteristic values) and in some instances corresponding textures for the texture characteristics found in prior or historical CT slices. For example, the texture characteristics can include a value or a range of values for a respective texture, such as coarse-grained, fine-grained, and shale texture. Thus, for each texture, the training data can include a value or ranges of values indicative of one of a grain size, shape, orientation, and/or pore structure for that texture. For example, for a coarse-grained texture, the texture characteristics can include a value or range of values indicative of a large grain size, a rounded shape, a grain orientation, and/or pore space connectivity. In some examples, the training dataset includes a texture label along with observed (or identified) characteristics (e.g., grain size, shape, orientation, and/or pore structure). By way of even further example, a texture label for a respective CT slice of the training dataset can include can indicate a grain size of 3 mm (e.g., corresponding to a large grain size), a rounded shape, a random orientation, a large, interconnected pore structure, and an identifying of the texture label, for example, “coarse-grained.” The ML algorithm can be trained using the training dataset, referred to as a training phase. For example, during the training phase, which can be implemented by the analysis engine 114, in some instances, the ML algorithm can be trained to learn to recognize (or identify) different textures. During training, the ML algorithm learns to associate specific patterns in the images with corresponding texture labels and in some instances texture values to provide the texture classifier 122.

For example, the texture classifier 122 can process the CT slices 136 of the core plug 106 to identify and label each texture (in some instances different textures) present in each CT slice of the CT slices 136, such as coarse-grained, fine-grained, or shale texture. The output from the texture classifier 122 can also provide values for texture characteristics for each texture, such as grain size, a shape, orientation and/or pore structure. The texture classifier 122 can provide texture data 124 for each CT slice that includes the texture label and values for that texture label for a corresponding CT slice. Thus, the texture data 124 can characterize textures present in each CT slice and associated texture characteristics.

In some examples, a saturation process 126 can be applied to the core plug 106 (e.g., in some instances after being cleaned). The saturation process 126 can include saturating the core plug 106 with brine to fill pore spaces of the core plug 106 with a liquid to simulate conditions of the reservoir 104. The conditions can include the presence of fluids such as water, oil, and/or gas within pore spaces (pore structure) of the core plug 106. By saturating the core plug 106 with brine, which mimics a fluid present in the reservoir 104, natural conditions of fluid saturation within the rock can be replicated for assessing properties of the core plug 106.

After saturation, the core plug 106 can be subjected to a nuclear magnetic resonance (NMR) measurement. For example, the core plug 106 can be analyzed by an NMR device 128 (also can be referred to as an NMR scanner). The NMR device 128 can be used to measure a response of an atomic nuclei in the core plug 106 to a magnetic field and radio-frequency (RF) pulses to provide NMR data 130 for determining permeability and porosity. The NMR device 128 conducts spatial measurements along the length of the core plug 106, characterizing the relaxation time distribution across different regions within the core plug 106. These measurements reflect variations in pore sizes within the core plug 106. The NMR data 130, in the form of a relaxation time spectrum, characterizes the pore size distribution. A relaxation time distribution represents the decay of an NMR signal over time.

Different types of pore spaces influence the relaxation process, resulting in a range of relaxation times. The amplitude of a signal in the relaxation time spectrum is proportional to the volume of fluid-filled pores, corresponding to porosity. By analyzing the relaxation time spectrum, the porosity of the core plug 106 can be estimated. The shape and characteristics of the relaxation time spectrum are used to estimate permeability. Finer pores exhibit shorter relaxation times, while larger pores have longer relaxation times. Therefore, the relaxation time spectrum can quantify the permeability of the core plug 106 and, by extension, the reservoir 104, after validation as disclosed.

In some examples, an NMR resolution of the NMR device 128 can be set to either 32 or 64 to set a level of detail of the spatial measurement. The NMR resolution can indicate a number of data points or intervals used in the spatial measurement. A higher resolution enables finer distinctions to be made in relaxation times across spatial locations within the core plug 106. Thus, with a resolution of 64, more detailed information about the distribution of the relaxation times (e.g., T² values) can be obtained compared to a resolution of 32.

In some examples, a FOV of the NMR device 128 can be set for analyzing or scanning the core plug 106. By way of example, the FOV can be set to about 6 to about 7 centimeters (cm). The FOV of the NMR device 128 refers to an extent of the core plug 106 that can be imaged in a single scan (e.g., measurement of relaxation times). The FOV of the NMR device 128 can be set so that a scan implemented by the NMR device 128 encompasses an entirety of the core plug 106 to ensure that NMR measurements cover the entire length of the core plug 106.

For example, a sampling interval can be defined for the NMR device 128. As an example, if the core plug 106 is 2 inches in length, an NMR measurement with a spatial resolution of 32 or 64, which can divide a length of the core plug 106 into equally spaced intervals to match a spatial resolution for the CT slices 136. For a FOV of about 6 to about 7 cm, each sampling interval can correspond to a length of about 3 to about 3.5 cm. The NMR device 128 can perform the NMR measurement on the core plug 106 to collect NMR signal responses (the relaxation times) at each sampling interval along the length of the core plug 106 to provide the NMR data 130. Accordingly, once the NMR measurement is complete, the NMR data 130 can contain NMR signal responses obtained from different parts of the core plug 106.

The analysis engine 114 includes an NMR segmentor 132. The NMR segmentor 132 can segment (divide) the NMR data 130 into NMR segments 144 (or NMR slices). For example, the NMR segmentor 132 can segment the NMR data 130 into intervals corresponding to each sampling interval along the length of the core plug 106 to provide NMR segments 144. A number of NMR segments can depend on the selected spatial resolution (32 or 64) and the length of the core plug 106. By way of example, a first NMR segment of the NMR segments 144 can include a first subset of the NMR data 130 that was collected from about 0 to about 1 cm along the length of the core plug 106. A second NMR segment of the NMR segments 144 can include a second subset of the NMR data 130 that was collected from about 1 cm to about 2 cm. For example, if the spatial resolution is 32, there can be 27 NMR segment as the NMR segments 144, and if the spatial resolution is 64 there can be 54 NMR segments as the NMR segments 144. Each NMR segment of the NMR segments 144 can include data points that are associated with a portion (segment) of the core plug 106. Thus, the NMR segments 144 are spatially mapped to a portion (segment) of the core plug 106 along its length 06. The NMR segments 144 can include information characterizing variations in relaxation times or signal responses across respective sections of the core plug 106 along its length and thus a thickness of the core plug 106. Data points used for representing each CT slice of the CT slices 36 of the core plug 106 along its length are spatially mapped to a similar sized portion (segment) of the core plug 106 along its length. Thus, the CT slices 136 (and thus the texture data 124 for each CT slice) are aligned spatially with the NMR segments 144.

The analysis engine 114 includes a factor analyzer 146. The factor analyzer 146 can apply an Exploratory Factor Analysis (EFA) using the NMR segments 144 and the texture data 124. Each texture identified in the texture data 124 has an associated unique combination of texture characteristics (e.g., grain size, shape, orientation, and/or pore structure). Each CT slice of the CT slices 136 can have multiple textures contributing to the relaxation times of the nuclei in the core plug 106, which are influenced by the texture and/or structure of the core plug 106. The factor analyzer 146 can output factors that correspond to T2 distribution peaks. A T2 distribution peak refers to a distinct peak or maximum point in a distribution of relaxation times. These peaks represent different types of pore sizes and structures within a material. The position and/or amplitude of a relaxation time (T2 distribution) peak provides information about the size and abundance of pores in the core plug 106. The factors are variables that characterize the variation in the observed NMR data, which is a subset of the NMR data 130 corresponding to a respective CT slice of the CT slices 136. Each texture (e.g., coarse-grained, fine-grained, etc.) has a unique combination of these factors, which translates into specific T2 distribution peaks.

The factor analyzer 146 can determine the contributions of each texture to the observed T2 distribution to provide detailed insights into the structure and/or properties of the core plug 106. For example, it can decompose the observed NMR data into contributions from different textures. The factor analyzer 146 can use factor analysis techniques to identify the contributions of each factor (texture characteristic) to observed T2 distribution peaks. For example, the factor analyzer 146 can use the following expression:

$$T {2}_{observed} = {\sum }_{i} {w}_{i} \times {F}_{i} , \qquad ( 1 )$$

wherein T2_{observed} is the observed T2 distribution for a particular CT slice, Fᵢ represents a factor corresponding to a specific texture (e.g., coarse-grained, fine-grained), and wᵢ are weights that indicate a contribution of each factor to the observed T2 distribution.

The factor analyzer 146 can output factors, weights, decomposed T2 distributions, and texture characteristics. Each factor outputted by the factor analyzer 146 corresponds to specific T2 distribution peaks that are characteristic of particular textures. The weights outputted by the factor analyzer 146 are the contributions of each factor to the observed T2 distribution. The weights indicate how much each texture (factor) contributes to the overall relaxation time distribution in a given CT slice. The factor analyzer 146 provides the decomposed T2 distributions for each texture and thus separates the observed T2 distribution into individual contributions from different textures. Thus, the factor analyzer 146 can identify the presence of different textures within each CT slice of the CT slices 136 by decomposing the observed T2 distribution, provide quantitative contributions (weights) of each identified texture to the observed T2 distribution, and identify specific T2 peaks associated with each texture.

Accordingly, the output of the EFA provides the factors or T2 distribution peaks, which correspond to different pore systems of different textures. The factor analyzer 146 can recover NMR T2 distributions for each texture within a CT slice. These peaks in the T2 distributions indicate the presence of various pore sizes and structures within the core plug 106, reflecting the unique characteristics of the textures. Collectively, the output of the factor analyzer 146 can be referred to as a texture relaxation time (T2 distribution) profile 138. The texture relaxation time profile 138 for each CT slice of the CT slices 136 characterizes a unique relaxation time (T2) distribution associated with different textures and thus provides information about pore sizes, texture identification, quantitative contributions, and structural insights into the core plug 106. Accordingly, the factor analyzer 146 uses EFA to decompose the observed NMR T2 distributions into contributions from different textures by leveraging the unique combination of factors associated with each texture.

The analysis engine 114 can use the factor analyzer 146 to validate text volumes for each NMR and CT slice using both microCT and NMR factor analysis. The factor analyzer 146 can confirm that the volume of textures from microCT images correspond to the volumes of NMR.

The analysis engine 114 can include a permeability calculator 142 to determine the permeability for each texture within each CT slice of the CT slices 136. For each CT slice, multiple permeability values can be calculated by the permeability calculator 142, each corresponding to a different texture present in that CT slice. The permeability can be calculated using the Coates or Schlumberger-Doll Research (SDR) models. These models provide methods to estimate permeability for each texture within each CT slice based on NMR relaxation data (a corresponding NMR segment of the NMR segments 144) and texture characteristics. The texture characteristics, as disclosed herein, are provided by the factor analyzer 146 as the texture relaxation time profile 138. The factor analyzer 146 uses EFA to identify these characteristics by analyzing the T2 distribution peaks from a corresponding NMR segment and CT slice. Each texture (e.g., coarse-grained, fine-grained, shale) is characterized by a unique combination of these attributes.

Each texture has its own unique correlation parameters, which are specific values or coefficients used in the models to improve the accuracy of permeability predictions. For example, the Coates model can use correlation parameters related to porosity and a specific surface area of textures, while the SDR model uses correlation parameters associated with NMR relaxation times and porosity. By applying these correlation parameters to the respective models, the permeability calculator 142 can predict permeability for each texture within the core plug 106. Thus, unique characteristics of different textures, such as coarse-grained, fine-grained, and shale, can be accounted for by the permeability calculator, which results in more accurate permeability estimates. The estimated permeability values can then be used to understand fluid flow properties within the reservoir, and thus aid in reservoir characterization and management.

For example, the SDR model, applicable to a 100% water saturated-sample, that can be used by the permeability calculator 142 can be expressed as follows:

$$k = a * {ϕ}^{m} {T}_{2 LM}^{2 n} , \qquad ( 2 )$$

wherein ϕ is porosity,

$${T}_{2 LM}^{2}$$

is a logarithmic mean of an NMR T² distribution, a, m, and n are correlation parameters, and k is a permeability of the rock sample.

For example, the correlation parameters in expression (2) can be specific to each texture type and can be adjusted to control the SDR model to more accurately predict permeability based on texture characteristics. For example, coarse-grained textures can have higher values for a and m to reflect their larger pore sizes and greater influence of porosity on permeability. The logarithmic mean of the T2 distribution

$$( {T}_{2 LM}^{2} )$$

of the core plug 106 averages values across different regions for heterogeneous core samples. While T² measurements can encompass an entire sample, spatial measurements are feasible if an instrument possesses a gradient. Achieving one-dimensional (1D) spatial T² distribution for each slice along a sample length in a reasonable timeframe is possible. The FOV and total slice count can be adjusted, typically settling at a slice thicknesses of 1 millimeter (mm), 2 mm, or 4 mm. Thus, 2D and 3D spatial T2 distributions can be secured. In some instances, for samples with varied textures, spatial T² distribution can discern differences along a sample length. Differentiation within slices can be based on a statistical method, such as EFA. EFA can be applied to analyze the T² distribution obtained from NMR data 130 to identify different fluid components within the core plug 106. In fully water-saturated samples, the changing T² distributions throughout a core length reflect textures can be observed within slices, as determined through deep learning analysis of microCT images, for example, by the texture classifier 122.

In some examples, porosity can be determined by the permeability calculator 142 from microCT if the carbonate is mainly composed of one mineral (calcite or dolomite). For example, high-resolution images from microCT scans (e.g., the core plug image 112) can be used to accurately capture the pore spaces within the rock matrix, and porosity is calculated by the permeability calculator 142 by dividing the volume of the pore spaces by the total volume of the rock sample. For complex mineral compositions, the approach can be applied similarly. The microCT data (e.g., the core plug image 112) is analyzed by the permeability calculator 142 to identify and segment different minerals within the core plug 106 using image processing techniques and machine learning algorithms. These minerals, or “mineral phases,” are distinct types of minerals present in the rock, such as calcite, dolomite, quartz, or clay. The segmented pore spaces can then be used to calculate a total porosity. In some laboratory settings, typically, only one T² distribution of the entire core sample (the core plug 106) is considered, assuming a sample is uniform throughout. Porosity can be determined by comparing a total measured water content to a core sample's overall volume. This method of porosity measurement can be used to capture all types of porosities, including microporosity.

In some examples, the analysis engine 114 includes a model generator 148 to provide a petrophysical model 150 based on the permeability (permeability values) and/or porosity (porosity values) computed for each texture within each CT slice of the CT slices 136. The model generator 148 can construct a continuum model, such as a Darcy-scale model, which can be provided as the petrophysical model 150. In some examples, the petrophysical model 150 includes a permeability and/or a porosity model. The Darcy-scale model can provide an approximation of an overall permeability and/or porosity behavior of the core plug 106 at a larger scale. The petrophysical model 150 can use permeability and porosity values provided by the permeability calculator 142 to create a larger-scale, simplified representation of the core plug 106. The petrophysical model 150 can predict fluid flow and reservoir behavior, optimize extraction strategies, and enhance reservoir management by providing a detailed understanding of the subsurface properties.

For example, the petrophysical model 150, implemented as a Darcy-scale model, can approximate fluid flow behavior within the core plug 106 at a larger scale by using Darcy's law to simulate how fluids such as oil, water, and/or gas move through a pore structure (or network) of the core plug 106. By incorporating permeability and porosity values, the petrophysical model 150 can be used to identify preferred flow paths and potential barriers within the core plug 106, with higher permeability zones indicating easier fluid movement and lower permeability zones highlighting areas of restricted flow. In some examples, the petrophysical model 150 can be used to predict a pressure distribution throughout the core plug 106 for designing effective extraction strategies and managing reservoir depletion. In some examples, the petrophysical model 150 can be used for estimating fluid saturation levels within different parts of a reservoir, providing information about a presence of oil, water, and/or gas, which can be used in assessing potential hydrocarbon recovery.

Additionally, the model aids in optimizing extraction strategies by informing the design of enhanced recovery techniques such as water flooding or gas injection, ensuring optimal placement of injection and production wells to maximize recovery. It also informs well placement and design decisions, ensuring wells are drilled and designed to maximize production and minimize risks. In terms of reservoir management, the model enables the prediction of future production rates and the overall recovery factor of the reservoir, aiding in the planning and optimization of production schedules. As production progresses, the model can be updated with new data to continuously refine predictions and adjust strategies, ensuring that operations remain efficient and adaptive to changing conditions.

For example, in a carbonate reservoir with heterogeneous textures including coarse-grained, fine-grained, and shale zones, the petrophysical model incorporates permeability and porosity values for these textures within each CT slice of the core plug. Engineers use this model to simulate fluid flow under various production scenarios such as primary depletion, water flooding, or gas injection. During primary depletion, the model predicts how pressure will decline over time and where fluids will move as production progresses. For water flooding, the model helps design the flood pattern by predicting how injected water will sweep oil towards production wells and identifying areas where water breakthroughs might occur prematurely. In gas injection scenarios, the model assists in optimizing gas injection rates and locations to maintain reservoir pressure and enhance oil recovery. By using the petrophysical model in these ways, engineers can make informed decisions that improve the efficiency and effectiveness of reservoir development and management, ultimately leading to better economic and operational outcomes.

In some examples, laboratory rock samples (e.g., the core plug 106) undergo cleaning and saturation before any subsequent measurements. Given the non-invasive nature of the methods, integrating NMR measurements of 100% water-saturated samples and microCT scans of dry, clean samples into the system 100 can be achieved. The derived continuum porosity/permeability models can augment other measurements, like displacement experiments, ensuring a more precise interpretation considering the core scale heterogeneity. Additionally, texture classification can serve as a core-scale step in upscaling heterogeneous carbonate properties. Most laboratory measurements and interpretation assume that the core is homogeneous, and the modeling is often done using a 1D linear model of uniform porosity and permeability. This traditional approach simplifies the analysis but overlooks the natural heterogeneity present in most rock formations. By using the petrophysical model 150, a better interpretation can be obtained as it provides a detailed, 3D representation of core plug properties, capturing spatial variations in porosity and/or permeability. With textures from multiple core samples and the potential combination with well logging data, it is possible to upscale from core scale to a larger scale. This integration enhances an accuracy of reservoir models, improves fluid flow predictions and aids in the optimization of extraction strategies and reservoir management.

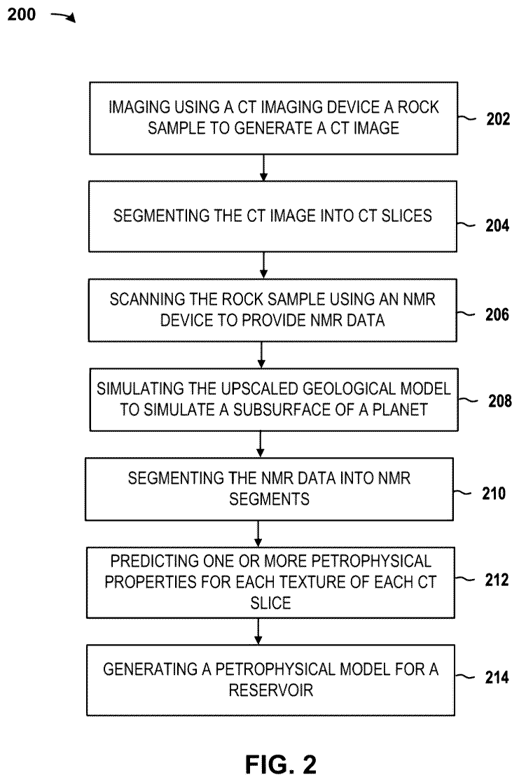

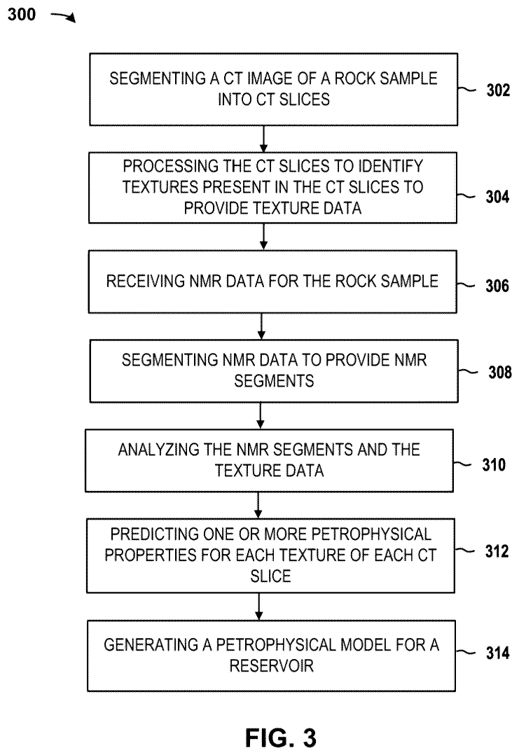

In view of the foregoing structural and functional features described above, example methods will be better appreciated with reference to FIGS. 2-3. While, for purposes of simplicity of explanation, the example methods of FIGS. 2-3 are shown and described as executing serially, it is to be understood and appreciated that the present example is not limited by the illustrated order, as some actions could in other examples occur in different orders, multiple times and/or concurrently from that shown and disclosed herein. Moreover, it is not necessary that all described actions be performed to implement the methods.

FIG. 2 is an example of a method 200 for generating petrophysical model (e.g., the petrophysical model 150, as shown in FIG. 1) for a reservoir (e.g., the reservoir 104, as shown in FIG. 1). One or more steps of the method 200 can be implemented by the tool 100, as shown in FIG. 1. Thus, reference can be made to one or more examples of FIG. 1 in the example of FIG. 2. The method 200 can begin at 202 by imaging, using a CT imaging device (e.g., the CT imaging device 110, as shown in FIG. 1) to generate a CT image (e.g., the core plug image 112, as shown in FIG. 1) of a rock sample (e.g., the core plug 106, as shown in FIG. 1) from the reservoir. At 204, the CT image is segmented (e.g., by the CT segmentor 134, as shown in FIG. 1) into CT slices (e.g., the CT slices 136, as shown in FIG. 1). At 206, the CT slices are processed using a texture classifier (e.g., the texture classifier 122, as shown in FIG. 1) to identify each texture present in each CT slice of the CT slices to provide texture data (e.g., the texture data 124, as shown in FIG. 1) for each CT slice. At 208, the rock sample is scanned using an NMR device (e.g., the NMR device 128, as shown in FIG. 1) to provide NMR data (e.g., the NMR data 130, as shown in FIG. 1) for the rock sample. The NMR data can characterize relaxation times across different regions of the rock sample reflecting variations in pore sizes within the rock sample. At 210, the NMR data is segmented (e.g., by the NMR segmentor 132, as shown in FIG. 1) into intervals corresponding to a scanning interval of a portion of the rock sample scanned by an NMR device (e.g., the NMR device 128, as shown in FIG. 1) to provide NMR segments (e.g., the NMR segments 144, as shown in FIG. 1). At 212, the NMR segments and the texture data are analyzed (e.g., by the factor analyzer 146, as shown in FIG. 1) to determine a contribution of each texture in each CT slice to one or more relaxation times in a corresponding NMR segment of the NMR segments for each CT slice. At 214, one or more petrophysical properties for each texture of each CT slice is predicted (e.g., by the permeability calculator 142, as shown in FIG. 1) based on a determined contribution of each texture and the corresponding NMR segment for each CT slice. At 216, the petrophysical model for the reservoir is generated (e.g., by the model generator 148, as shown in FIG. 1) based on the predicted one or more petrophysical properties for each texture of each CT slice.

FIG. 3 is an example of a method 300 for generating petrophysical model (e.g., the petrophysical model 150, as shown in FIG. 1) for a reservoir (e.g., the reservoir 104, as shown in FIG. 1). The method 300 can be implemented by the analysis engine 114, as shown in FIG. 1. Thus, reference can be made to one or more examples of FIG. 1 in the example of FIG. 3. The method 300 can begin at 302 with a CT image (e.g., the core plug image 112, as shown in FIG. 1) of a rock sample (e.g., the core plug 106, as shown in FIG. 1) from the reservoir being segmented (e.g., by the CT segmentor 134, as shown in FIG. 1) into CT slices (e.g., the CT slices 136, as shown in FIG. 1). At 304 the CT slices can be processed (e.g., by the texture classifier 122, as shown in FIG. 1) to identify each texture present in each CT slice of the CT slices to provide texture data (e.g., the texture data 124, as shown in FIG. 1) for each CT slice. At 306, NMR data (e.g., the NMR data 130, as shown in FIG. 1) generated from an NMR scan of the rock sample can be received (e.g., by the NMR segmentor 132, as shown in FIG. 1). The NMR data can characterize relaxation times across different regions of the rock sample reflecting variations in pore sizes within the rock sample. At 308, the NMR data is segmented (e.g., by the NMR segmentor 132) into intervals corresponding to a scanning interval of a portion of the rock sample by an NMR device (e.g., the NMR device 128, as shown in FIG. 1) to provide NMR segments (e.g., the NMR segments 144, as shown in FIG. 1). At 310, the NMR segments and the texture data are analyzed (e.g., by the factor analyzer 146, as shown in FIG. 1) to determine a contribution of each texture in each CT slice to one or more relaxation times in a corresponding NMR segment of the NMR segments for each CT slice. At 312, one or more petrophysical properties for each texture of each CT slice are determined (e.g., by the permeability calculator 142, as shown in FIG. 1) based on a determined contribution of each texture and the corresponding NMR segment for each CT slice. At 314, the petrophysical model for the reservoir is generated (e.g., by the model generator 148, as shown in FIG. 1) based on the predicted one or more petrophysical properties for each texture of each CT slice.

### Additional Exemplary Embodiments

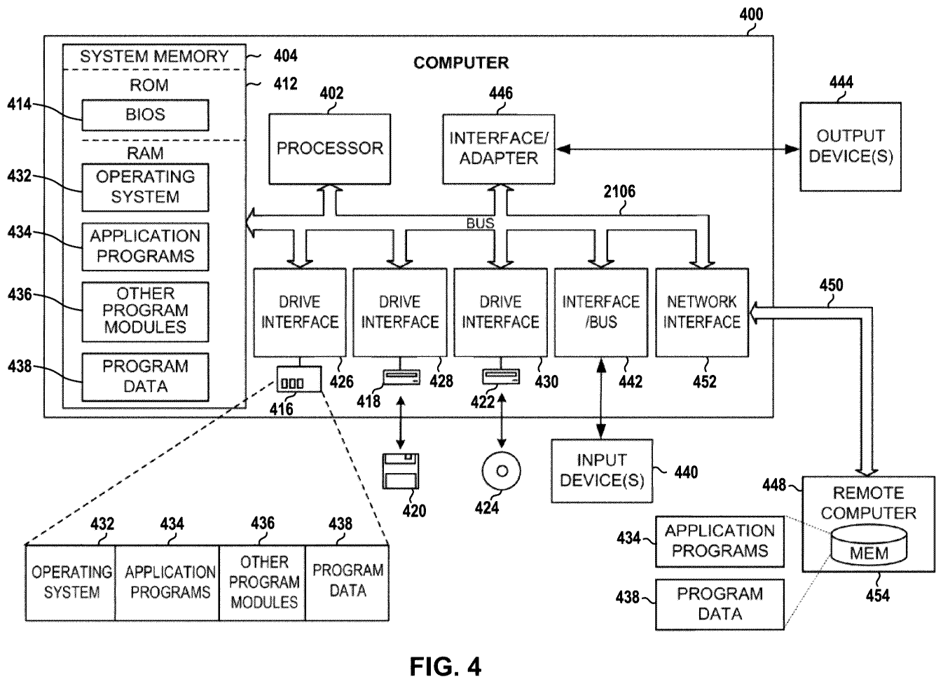

In view of the foregoing structural and functional description, those skilled in the art will appreciate that portions of the embodiments may be embodied as a method, data processing system, or computer program product. Accordingly, these portions of the present embodiments may take the form of an entirely hardware embodiment, an entirely software embodiment, or an embodiment combining software and hardware, such as shown and described with respect to the computer system of FIG. 4. Thus, reference can be made to one or more examples of FIGS. 1-3 in the example of FIG. 4.

In this regard, FIG. 4 illustrates one example of a computer system 400 that can be employed to execute one or more embodiments of the present disclosure. Computer system 400 can be implemented on one or more general purpose networked computer systems, embedded computer systems, routers, switches, server devices, client devices, various intermediate devices/nodes, or standalone computer systems. Additionally, computer system 400 can be implemented on various mobile clients such as, for example, a personal digital assistant (PDA), laptop computer, pager, and the like, provided it includes sufficient processing capabilities.

Computer system 400 includes processing unit 402, system memory 404, and system bus 406 that couples various system components, including the system memory 404, to processing unit 402. Dual microprocessors and other multi-processor architectures also can be used as processing unit 402. System bus 406 may be any of several types of bus structure including a memory bus or memory controller, a peripheral bus, and a local bus using any of a variety of bus architectures. System memory 404 includes read only memory (ROM) 410 and random access memory (RAM) 412. A basic input/output system (BIOS) 414 can reside in ROM 412 containing the basic routines that help to transfer information among elements within computer system 400.

Computer system 400 can include a hard disk drive 416, magnetic disk drive 418, e.g., to read from or write to removable disk 420, and an optical disk drive 422, e.g., for reading CD-ROM disk 424 or to read from or write to other optical media. Hard disk drive 416, magnetic disk drive 418, and optical disk drive 422 are connected to system bus 406 by a hard disk drive interface 426, a magnetic disk drive interface 428, and an optical drive interface 430, respectively. The drives and associated computer-readable media provide nonvolatile storage of data, data structures, and computer-executable instructions for computer system 400. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of embodiments shown and disclosed herein. A number of program modules may be stored in drives and RAM 410, including operating system 432, one or more application programs 434, other program modules 436, and program data 438. In some examples, the application programs 434 can include one or more modules (or block diagrams), or systems, as shown and disclosed herein. Thus, in some examples, the application programs 434 can include the analysis engine 114, as shown in FIG. 1. In some examples, the application programs 434 includes software, and the analysis engine 114 can be implemented as part of the software, or interact with the software.

A user may enter commands and information into computer system 400 through one or more input devices 440, such as a pointing device (e.g., a mouse, touch screen), keyboard, microphone, joystick, game pad, scanner, and the like. These and other input devices are often connected to processing unit 402 through a corresponding port interface 442 that is coupled to the system bus, but may be connected by other interfaces, such as a parallel port, serial port, or universal serial bus (USB). One or more output devices 444 (e.g., display, a monitor, printer, projector, or other type of displaying device) is also connected to system bus 406 via interface 446, such as a video adapter.

Computer system 400 may operate in a networked environment using logical connections to one or more remote computers, such as remote computer 448. Remote computer 448 may be a workstation, computer system, router, peer device, or other common network node, and typically includes many or all the elements described relative to computer system 400. The logical connections, schematically indicated at 450, can include a local area network (LAN) and a wide area network (WAN). When used in a LAN networking environment, computer system 400 can be connected to the local network through a network interface or adapter 452. When used in a WAN networking environment, computer system 400 can include a modem, or can be connected to a communications server on the LAN. The modem, which may be internal or external, can be connected to system bus 406 via an appropriate port interface. In a networked environment, application programs 434 or program data 438 depicted relative to computer system 400, or portions thereof, may be stored in a remote memory storage device 454.

Although this disclosure includes a detailed description on a computing platform and/or computer, implementation of the teachings recited herein are not limited to only such computing platforms. Rather, embodiments of the present disclosure are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models (e.g., software as a service (Saas, platform as a service (PaaS), and/or infrastructure as a service (IaaS)) and at least four deployment models (e.g., private cloud, community cloud, public cloud, and/or hybrid cloud). A cloud computing environment can be service oriented with a focus on statelessness, low coupling, modularity, and semantic interoperability.

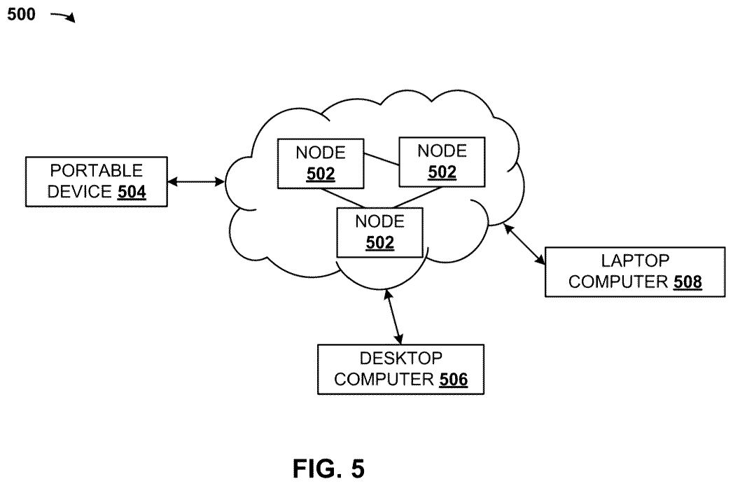

FIG. 5 is an example of a cloud computing environment 500 that can be used for implementing one or more modules and/or systems in accordance with one or more examples, as disclosed herein. Thus, reference can be made to one or more examples of FIGS. 1-5 in the example of FIG. 5. As shown, cloud computing environment 500 can include one or more cloud computing nodes 502 with which local computing devices used by cloud consumers (or users), such as, for example, personal digital assistant (PDA), cellular, or portable device 504, a desktop computer 506, and/or a laptop computer 508, may communicate. The computing nodes 502 can communicate with one another. In some examples, the computing nodes 502 can be grouped (not shown) physically or virtually, in one or more networks, such as Private, Community, Public, or Hybrid clouds, or a combination thereof. This allows the cloud computing environment 500 to offer infrastructure, platforms and/or software as services for which a cloud consumer does not need to maintain resources on a local computing device. The devices 504-508, as shown in FIG. 5, are intended to be illustrative and that computing nodes 502 and cloud computing environment 500 can communicate with any type of computerized device over any type of network and/or network addressable connection (e.g., using a web browser). In some examples, the one or more computing nodes 802 are used for implementing one or more examples disclosed herein relating to root-source identification. Thus, in some examples, the one or more computing nodes can be used to implement modules, platforms, and/or systems, as disclosed herein.

In some examples, the cloud computing environment 500 can provide one or more functional abstraction layers. It is to be understood that the cloud computing environment 500 need not provide all of the one or more functional abstraction layers (and corresponding functions and/or components), as disclosed herein. For example, the cloud computing environment 500 can provide a hardware and software layer that can include hardware and software components. Examples of hardware components include mainframes; RISC (Reduced Instruction Set Computer) architecture based servers; servers; blade servers; storage devices; and networks and networking components. In some embodiments, software components include network application server software and database software.

In some examples, the cloud computing environment 500 can provide a virtualization layer that provides an abstraction layer from which the following examples of virtual entities may be provided: virtual servers; virtual storage; virtual networks, including virtual private networks; virtual applications and operating systems; and virtual clients. In some examples, the cloud computing environment 500 can provide a management layer that can provide the functions described below. For example, the management layer can provide resource provisioning that can provide dynamic procurement of computing resources and other resources that are utilized to perform tasks within the cloud computing environment. The management layer can also provide metering and pricing to provide cost tracking as resources are utilized within the cloud computing environment 500, and billing or invoicing for consumption of these resources. In one example, these resources may include application software licenses. Security provides identity verification for cloud consumers and tasks, as well as protection for data and other resources. The management layer can also provide a user portal that provides access to the cloud computing environment 500 for consumers and system administrators. The management layer can also provide service level management, which can provide cloud computing resource allocation and management such that required service levels are met. Service Level Agreement (SLA) planning and fulfillment can also be provided to provide pre-arrangement for, and procurement of, cloud computing resources for which a future requirement is anticipated in accordance with an SLA.

In some examples, the cloud computing environment 500 can provide a workloads layer that provides examples of functionality for which the cloud computing environment 500 may be utilized. Examples of workloads and functions which may be provided from this layer include mapping and navigation; software development and lifecycle management; virtual classroom education delivery; data analytics processing; and transaction processing. Various embodiments of the present disclosure can utilize the cloud computing environment 500.

The present disclosure is also directed to the following exemplary embodiments, which can be practiced in any combination thereof:

Embodiment A: a method comprising: imaging, using a CT imaging device to generate a CT image of a rock sample from a reservoir, segmenting the CT image into CT slices; processing the CT slices using a texture classifier to identify textures of the rock sample to provide texture data for each CT slice; scanning the rock sample using an NMR device to provide NMR data for the rock sample, the NMR data characterizing relaxation times across different regions of the rock sample reflecting variations in pore sizes within the rock sample; segmenting the NMR data into intervals corresponding to a scanning interval of a portion of the rock sample by the NMR device to provide NMR segments; analyzing the NMR segments and the texture data to determine a contribution of each texture in each CT slice to one or more relaxation times in a corresponding NMR segment of the NMR segments for each CT slice; predicting one or more petrophysical properties for each texture of each CT slice based on a determined contribution of each texture and the corresponding NMR segment for each CT slice; and generating a petrophysical model for the reservoir based on the predicted one or more petrophysical properties for each texture of each CT slice.

Embodiment B: A system comprising one or more computing platforms configured to: segment a CT image of a rock sample from a reservoir into CT slices; process the CT slices using a texture classifier to identify textures of the rock sample to provide texture data for each CT slice; receive NMR data generated from an NMR scan of the rock sample, the NMR data characterizing relaxation times across different regions of the rock sample reflecting variations in pore sizes within the rock sample; segment the NMR data into intervals corresponding to a scanning interval of a portion of the rock sample scanned by an NMR device to provide NMR segments; analyze the NMR segments and the texture data to determine a contribution of each texture in each CT slice to one or more relaxation times in a corresponding NMR segment of the NMR segments for each CT slice; determine one or more petrophysical properties for each texture of each CT slice based on a determined contribution of each texture and the corresponding NMR segment for each CT slice; and generate a petrophysical model for the reservoir based on the predicted one or more petrophysical properties for each texture of each CT slice.

Embodiment 3: A system comprising: memory to store machine-readable instructions; one or more processors to access the memory and execute the machine-readable instructions, the machine-readable instructions comprising: a CT image segmentor to segment a CT image of a rock sample from a reservoir into CT slices; a texture classifier to identify textures of the rock sample to provide texture data for each CT slice; an NMR image segmentor to segment NMR data into intervals corresponding to a scanning interval of a portion of the rock sample scanned by an NMR device to provide NMR segments; a factor analyzer to analyze the NMR segments and the texture data to determine a contribution of each texture in each CT slice to one or more relaxation times in a corresponding NMR segment of the NMR segments for each CT slice; a calculator to determine one or more petrophysical properties for each texture of each CT slice based on a determined contribution of each texture and the corresponding NMR segment for each CT slice; and a model generator to generate a petrophysical model for the reservoir based on the predicted one or more petrophysical properties for each texture of each CT slice.

Each of embodiments A through C may have one or more of the following additional elements in any combination: Embodiment 1: wherein the imaging comprises scanning using the CT imaging the device to generate the image of the rock sample according to scanning parameters, the scanning parameters identifying a scanning resolution; Embodiment 2: wherein the segmenting the CT images is based on segmentation criteria, the segmentation criteria defines a volume of each segment that is segmented from the CT image to provide a corresponding CT slice of the CT slices; Embodiment 3: wherein a thickness of each CT slice of the CT slices is same or similar to a thickness to the corresponding NMR segment of the NMR segments; Embodiment 4: wherein the texture classifier is trained based on a training dataset that comprises texture characteristics and a texture of textures for each of the texture characteristics, the texture characteristics including a value or a range of values indicative of one or more of a grain size, shape, orientation, and pore structure for the texture, and the textures including a coarse-grained texture, fine-grained texture, and shale texture; Embodiment: 5: saturating the rock sample to fill pore spaces of the rock sample with a liquid to simulate conditions of the reservoir to provide a saturated rock sample, the NMR device being used to analyze the saturated rock sample to provide the NMR data; Embodiment 6: wherein the analyzing comprises using EFA to determine the contributions; Embodiment 7: wherein the one or more petrophysical properties is estimated using a Coates or SDR model; Embodiment 8: wherein the petrophysical model is a continuum model; Embodiment 9: using the petrophysical model to predict a fluid flow and/or a behavior of the reservoir; Embodiment 10: wherein the CT image is segmented based on segmentation criteria, the segmentation criteria defines a volume of each segment that is segmented from the CT image to provide a corresponding CT slice of the CT slices; Embodiment 11: wherein the texture classifier is trained based on a training dataset that comprises texture characteristics and a texture of textures for each of the texture characteristics; Embodiment 12: wherein the CT image of the rock is provided by a micro-CT scanner and the NMR data is provided by an NMR device; Embodiment 13: wherein the one or more computing platforms are further configured to predict a fluid flow and/or a behavior of the reservoir using the petrophysical model; and Embodiment 14: wherein the petrophysical model is used to predict a fluid flow and/or a behavior of the reservoir.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware based systems that perform the specified functions or acts or carry out combinations of special purpose hardware and computer instructions.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof. In addition, the use of ordinal numbers (e.g., first, second, third, etc.) is for distinction and not counting. For example, the use of “third” does not imply there must be a corresponding “first” or “second.” Also, as used herein, the terms “coupled” or “coupled to” or “connected” or “connected to” or “attached” or “attached to” may indicate establishing either a direct or indirect connection, and is not limited to either unless expressly referenced as such. Furthermore, to the extent that the terms “includes,” “has,” “possesses,” and the like are used in the detailed description, claims, appendices, and drawings such terms are intended to be inclusive in a manner similar to the term “comprising” as “comprising” is interpreted when employed as a transitional word in a claim. The term “based on” means “based at least in part on.” The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 5-10% of the indicated number.

What has been described above includes mere examples of systems, computer program products and computer-implemented methods. It is, of course, not possible to describe every conceivable combination of components, products and/or computer-implemented methods for purposes of describing this disclosure, but one of ordinary skill in the art can recognize that many further combinations and permutations of this disclosure are possible. The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

The invention claimed is:

1. A method comprising:
imaging, using a computed tomography (CT) imaging device to generate a CT image of a rock sample from a reservoir;
segmenting the CT image into CT slices;
processing the CT slices using a texture classifier to identify textures of the rock sample to provide texture data for each CT slice;
scanning the rock sample using an nuclear magnetic resonance (NMR) device to provide NMR data for the rock sample, the NMR data characterizing relaxation times across different regions of the rock sample reflecting variations in pore sizes within the rock sample;
segmenting the NMR data into intervals corresponding to a scanning interval of a portion of the rock sample by the NMR device to provide NMR segments;
analyzing the NMR segments and the texture data to determine a contribution of each texture in each CT slice to one or more relaxation times in a corresponding NMR segment of the NMR segments for each CT slice;
predicting one or more petrophysical properties for each texture of each CT slice based on a determined contribution of each texture and the corresponding NMR segment for each CT slice; and
generating a petrophysical model for the reservoir based on the predicted one or more petrophysical properties for each texture of each CT slice.

2. The method of claim 1, wherein the imaging comprises scanning using the CT imaging the device to generate the image of the rock sample according to scanning parameters, the scanning parameters identifying a scanning resolution.

3. The method of claim 1, wherein the segmenting the CT images is based on segmentation criteria, the segmentation criteria defines a volume of each segment that is segmented from the CT image to provide a corresponding CT slice of the CT slices.

4. The method of claim 1, wherein a thickness of each CT slice of the CT slices is same or similar to a thickness to the corresponding NMR segment of the NMR segments.

5. The method of claim 1, wherein the texture classifier is trained based on a training dataset that comprises texture characteristics and a texture of textures for each of the texture characteristics, the texture characteristics including a value or a range of values indicative of one or more of a grain size, shape, orientation, and pore structure for the texture, and the textures including a coarse-grained texture, fine-grained texture, and shale texture.

6. The method of claim 1, saturating the rock sample to fill pore spaces of the rock sample with a liquid to simulate conditions of the reservoir to provide a saturated rock sample, the NMR device being used to analyze the saturated rock sample to provide the NMR data.

7. The method of claim 1, wherein the analyzing comprises using Exploratory Factor Analysis (EFA) to determine the contributions.

8. The method of claim 1, wherein the one or more petrophysical properties is estimated using a Coates or Schlumberger-Doll Research (SDR) model.

9. The method of claim 1, wherein the petrophysical model is a continuum model.

10. The method of claim 1, using the petrophysical model to predict a fluid flow and/or a behavior of the reservoir.

11. A system comprising:
one or more computing platforms configured to:
segment a computed tomography (CT) image of a rock sample from a reservoir into CT slices;
process the CT slices using a texture classifier to identify textures of the rock sample to provide texture data for each CT slice;
receive nuclear magnetic resonance (NMR) data generated from an NMR scan of the rock sample, the NMR data characterizing relaxation times across different regions of the rock sample reflecting variations in pore sizes within the rock sample;
segment the NMR data into intervals corresponding to a scanning interval of a portion of the rock sample scanned by an NMR device to provide NMR segments;
analyze the NMR segments and the texture data to determine a contribution of each texture in each CT slice to one or more relaxation times in a corresponding NMR segment of the NMR segments for each CT slice;
determine one or more petrophysical properties for each texture of each CT slice based on a determined contribution of each texture and the corresponding NMR segment for each CT slice; and
generate a petrophysical model for the reservoir based on the predicted one or more petrophysical properties for each texture of each CT slice.

12. The system of claim 11, wherein the CT image is segmented based on segmentation criteria, the segmentation criteria defines a volume of each segment that is segmented from the CT image to provide a corresponding CT slice of the CT slices.

13. The system of claim 11, wherein the texture classifier is trained based on a training dataset that comprises texture characteristics and a texture of textures for each of the texture characteristics.

14. The system of claim 11, wherein the analysis of the NMR segments and the texture data is done using Exploratory Factor Analysis (EFA).

15. The system of claim 11, wherein the one or more petrophysical properties is determined using a Coates or Schlumberger-Doll Research (SDR) model.

16. The system of claim 1, wherein the CT image of the rock is provided by a micro-CT scanner and the NMR data is provided by an NMR device.

17. The system of claim 11, wherein the one or more computing platforms are further configured to predict a fluid flow and/or a behavior of the reservoir using the petrophysical model.

18. The system of claim 11, wherein a thickness of each CT slice of the CT slices is same or similar to a thickness to the corresponding NMR segment of the NMR segments.

19. A system comprising:
memory to store machine-readable instructions;
one or more processors to access the memory and execute the machine-readable instructions, the machine-readable instructions comprising:
a computed tomography (CT) image segmentor to segment a CT image of a rock sample from a reservoir into CT slices;
a texture classifier to identify textures of the rock sample to provide texture data for each CT slice;
an nuclear magnetic resonance (NMR) image segmentor to segment NMR data into intervals corresponding to a scanning interval of a portion of the rock sample scanned by an NMR device to provide NMR segments;
a factor analyzer to analyze the NMR segments and the texture data to determine a contribution of each texture in each CT slice to one or more relaxation times in a corresponding NMR segment of the NMR segments for each CT slice;
a calculator to determine one or more petrophysical properties for each texture of each CT slice based on a determined contribution of each texture and the corresponding NMR segment for each CT slice; and
a model generator to generate a petrophysical model for the reservoir based on the predicted one or more petrophysical properties for each texture of each CT slice.

20. The system of claim 19, wherein the petrophysical model is used to predict a fluid flow and/or a behavior of the reservoir.
